(19) United States
(12) Patent Application Publication
(10) Pub. No.: US 2007/0277552 A1
(43) Pub. Date: Dec. 6, 2007

(54) PROCESS FOR PRODUCING LIQUEFIED PETROLEUM GAS

(76) Inventors: Kaoru FUJIMOTO, Kitakyushu-shi (JP); Kenji ASAMI, Kitakyushu-shi (JP); Xiaohong LI, Kitakyushu-shi (JP); Qianwen ZHANG, Kitakyushu-shi (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/673,067

(22) Filed: Feb. 9, 2007

(30) Foreign Application Priority Data
Feb. 10, 2006 (JP) 2006-33622

(51) Int. Cl. C07C 1/04

(52) U.S. Cl. 62/618; 518/713

(57) ABSTRACT

A hydrocarbon gas containing propane or butane as a main component, i.e., a liquefied petroleum gas is produced by passing a raw material gas comprising at least one selected from the group consisting of methanol and dimethyl ether, and a synthesis gas through a catalyst layer comprising a catalyst for producing a liquefied petroleum gas.

### TECHNICAL FIELD

This invention relates to a process for producing a liquefied petroleum gas containing propane or butane as a main component from methanol and/or dimethyl ether.

### BACKGROUND ART

Liquefied petroleum gas (LPG) is a liquefied petroleum-based or natural-gas-based hydrocarbon which is gaseous at an ambient temperature under an atmospheric pressure by compression while optionally cooling, and the main component of it is propane or butane. LPG is advantageously transportable because it can be stored or transported in a liquid form. Thus, in contrast with a natural gas that requires a pipeline for supply, it has a characteristic that it can be filled in a container to be supplied to any place. For that reason, LPG comprising propane as a main component, i.e., propane gas has been widely used as a fuel for household and business use. At present, propane gas is supplied to about 25 million households (more than 50% of the total households) in Japan. In addition to household and business use, LPG is used as a fuel for a portable product such as a portable gas burner and a disposable lighter (mainly, butane gas), an industrial fuel and an automobile fuel.

Conventionally, LPG has been produced by 1) collection from a wet natural gas, 2) collection from a stabilization (vapor-pressure regulating) process of crude petroleum, 3) separation and extraction of a product in, for example, a petroleum refining process, or the like.

LPG, in particular propane gas used as a household/business fuel, can be expected to be in great demand in the future. Thus, it may be very useful to establish an industrially practicable and new process for producing LPG.

As a process for producing LPG, Japanese Patent Laid-open Publication No. 61-23688 discloses that a synthesis gas consisting of hydrogen and carbon monoxide is reacted in the presence of a mixed catalyst obtained by physically mixing a methanol synthesis catalyst such as a Cu—Zn-based catalyst, a Cr—Zn-based catalyst and a Pd-based catalyst, specifically a CuO—ZnO—Al₂O₃ catalyst or a Pd/SiO₂ catalyst with a methanol conversion catalyst composed of a zeolite having an average pore size of about 10 Å (1 nm) or more, specifically a Y-type zeolite, to give a liquefied petroleum gas or a mixture of hydrocarbons similar in composition to LPG.

Furthermore, as a process for producing LPG, “Selective Synthesis of LPG from Synthesis Gas”, Kaoru Fujimoto et al., Bull. Chem. Soc. Jpn., 58, p. 3059-3060 (1985) discloses that, using a hybrid catalyst consisting of a methanol synthesis catalyst such as a 4 wt % Pd/SiO₂, a Cu—Zn—Al mixed oxide {Cu:Zn:Al=40:23:37 (atomic ratio)} or a Cu-based low-pressure methanol synthesis catalyst (Trade name: BASF S3-85) and a high-silica Y-type zeolite with SiO₂/Al₂O₃=7.6 treated with steam at 450° C. for 1 hour, C2 to C4 paraffins can be produced in a selectivity of 69 to 85% via methanol and dimethyl ether from a synthesis gas.

On the other hand, “Methanol/Dimethyl Ether Conversion on Zeolite Catalysts for Indirect Synthesis of LPG from Natural Gas”, Yingjie Jin et al., Dai 92 Kai Shokubai Touronkai TouronkaiA Yokousyuu, (the summaries of the 92th Catalysis Society of Japan (CatSJ) Meeting, Meeting-A), p. 322, Sep. 18, 2003 discloses a process for producing LPG, using at least one selected from the group consisting of methanol and dimethyl ether as a starting material. Specifically, a starting gas, whose composition is methanol:H₂:N₂=1:1:1, was passed through the two-layered catalyst layer consisting of ZSM-5 as the former layer and Pt—C as the latter layer (ZSM-5/Pt—C Series) or a mixed catalyst layer consisting of ZSM-5 and Pt—C (ZSM-5/Pt—C Pellet-mixture), under a slightly increased pressure, at a reaction temperature of 603 K (330° C.) and at a methanol-based LHSV of 20 h⁻¹, to carry out an LPG production reaction.

Furthermore, “Selective Synthesis of LPG from DME”, Kenji Asami et al., Sekiyugakkai Dai 47 Kai Nenkai Jusyoukouen, Dai 53 Kai Kenkyuhappyoukai Kouenyoushi, (the summaries of the 47th annual meeting of the Japan Petroleum Institute), p. 98-99, May 19, 2004 discloses a process for producing LPG from dimethyl ether and hydrogen by a catalytic reaction. The catalyst used in the process is a hybrid catalyst consisting of a methanol synthesis catalyst and a zeolite (Cu—Zn/USY and the like), Pd ion-exchanged ZSM-5 (Pd-ZSM-5), Pt ion-exchanged ZSM-5 (Pt-ZSM-5), or the like. “Synthesis of LPG from DME with VIIIB Metal Supported on ZSM-5”, Kenji Asami et al., Dai 13 Kai Nihon Energy Gakkai Taikai Kouenyoushisyuu, (the summaries of the 13th meeting of the Japan Institute of Energy), p. 128-129, Jul. 29, 2004 discloses a process for producing LPG from dimethyl ether and hydrogen, using a VIIIB Metal Supported on ZSM-5, specifically Pd ion-exchanged ZSM-5 (Pd-ZSM-5), Pt ion-exchanged ZSM-5 (Pt-ZSM-5), or the like, as a catalyst.

### DISCLOSURE OF THE INVENTION

An objective of this invention is to provide a process for economically producing a hydrocarbon containing propane or butane as a main component, i.e., a liquefied petroleum gas (LPG) from methanol and/or dimethyl ether.

The present invention provides a process for producing a liquefied petroleum gas containing propane or butane as a main component, comprising a step of:

producing a liquefied petroleum gas by passing a raw material gas comprising at least one selected from the group consisting of methanol and dimethyl ether, hydrogen, and carbon monoxide through a catalyst layer comprising a catalyst for producing a liquefied petroleum gas.

Furthermore, the present invention provides a process for producing a liquefied petroleum gas containing propane or butane as a main component, comprising a step of:

producing a liquefied petroleum gas by passing a raw material gas comprising at least one selected from the group consisting of methanol and dimethyl ether, and a synthesis gas through a catalyst layer comprising a catalyst for producing a liquefied petroleum gas.

And, the present invention also provides a process for producing a liquefied petroleum gas, comprising:

(I) a step of producing a synthesis gas from a carbon-containing starting material and at least one selected from the group consisting of H₂O, O₂ and CO₂;

(II) a step of producing methanol wherein a methanol-containing gas is produced from the synthesis gas obtained in the step of producing a synthesis gas, using a methanol synthesis catalyst; and

(II) a step of producing a liquefied petroleum gas wherein a liquefied petroleum gas containing propane or butane as a main component is produced from the methanol-containing gas obtained in the step of producing methanol and the synthesis gas obtained in the step of producing a synthesis gas, using a catalyst for producing a liquefied petroleum gas.

Furthermore, the present invention provides a process for producing a liquefied petroleum gas, comprising:

(I) a step of producing a synthesis gas from a carbon-containing starting material and at least one selected from the group consisting of H₂O, O₂ and CO₂;

(II) a step of producing dimethyl ether wherein a dimethyl ether-containing gas is produced from the synthesis gas obtained in the step of producing a synthesis gas, using a dimethyl ether synthesis catalyst; and

(II) a step of producing a liquefied petroleum gas wherein a liquefied petroleum gas containing propane or butane as a main component is produced from the dimethyl ether-containing gas obtained in the step of producing dimethyl ether and the synthesis gas obtained in the step of producing a synthesis gas, using a catalyst for producing a liquefied petroleum gas.

Herein, “synthesis gas” means a mixed gas comprising hydrogen and carbon monoxide produced from a carbon-containing starting material including a natural gas and a coal, and is not limited to a mixed gas consisting of hydrogen and carbon monoxide. A synthesis gas may be, for example, a mixed gas comprising carbon dioxide, water, methane, ethane, ethylene and so on. A synthesis gas produced by reforming a natural gas generally contains, in addition to hydrogen and carbon monoxide, carbon dioxide and water vapor.

A hydrocarbon gas containing propane or butane as a main component, i.e., a liquefied petroleum gas (LPG) can be produced by reacting methanol and/or dimethyl ether with hydrogen. A catalyst for producing a liquefied petroleum gas used for this reaction comprises an olefin-hydrogenation catalyst component including Fe, Co, Ni, Cu, Zn, Ru, Rh, Pd, Ir and Pt, and a zeolite. Examples of a catalyst for producing a liquefied petroleum gas include Pd and/or Pt supported on ZSM-5 or USY-type zeolite, and a mixed catalyst comprising a Pd-based catalyst component in which Pd is supported on a support (for example, silica) and a USY-type zeolite.

A process for producing an olefin by reacting methanol and/or dimethyl ether in the presence of a zeolite catalyst has now been well known. In this reaction, however, a zeolite catalyst is apt to be deteriorated due to coking, and therefore, the catalyst may not have a sufficiently long catalyst life. It is also known that a zeolite catalyst may be generally more deteriorated when carbon monoxide and/or carbon dioxide is present in a reaction atmosphere.

In contrast to the above process for producing an olefin, a marked deterioration of a zeolite catalyst may not occur in the process for producing a paraffin from methanol and/or dimethyl ether using a zeolite catalyst, although paraffin and olefin, the products of these two processes, are both hydrocarbons. In the process for producing a paraffin of this invention, no problem may arise when carbon monoxide and/or carbon dioxide is present in a reaction atmosphere; rather a higher yield of paraffin is achieved when carbon monoxide is present in a reaction atmosphere.

According to the process of this invention wherein propane and/or butane is produced by reacting methanol and/or dimethyl ether with hydrogen, even though using a zeolite-containing catalyst, deterioration of a zeolite due to coking can be prevented and thus LPG can be stably produced for a long period with reducing a catalyst cost.

In addition, according to this invention, the amount of hydrogen to be used (the content of hydrogen in the starting gas) can be reduced. When LPG is produced from methanol and/or dimethyl ether, and hydrogen, water generates as a by-product. Water reacts with carbon monoxide to form hydrogen and carbon dioxide. The reaction is referred to as a shift reaction (CO+H₂O→CO₂+H₂). In this invention, an LPG production reaction is conducted in the presence of carbon monoxide. Consequently, hydrogen generates by the reaction of water, which is a by-product, and carbon monoxide while reacting. Therefore, in a case where the content of hydrogen in the starting gas is low, a sufficient amount of hydrogen is present in a reaction atmosphere while the LPG production reaction proceeds, and thus propane and/or butane can be produced with a high yield.

Furthermore, according to this invention, a synthesis gas can be used as the source of hydrogen, instead of pure hydrogen, for the reason that no problem may arise when carbon monoxide and/or carbon dioxide is present in a reaction atmosphere. A synthesis gas is inexpensive as compared with pure hydrogen. When using a synthesis gas comprising carbon monoxide, the yield of propane and/or butane may be approximately equal to the yield when using pure hydrogen. In addition, a synthesis gas is a raw material gas for methanol or dimethyl ether. Therefore, according to this invention, the unreacted raw material for the methanol synthesis reaction or the dimethyl ether synthesis reaction can be used as the source of hydrogen. Thus, according to this invention, LPG can be produced from methanol and/or dimethyl ether at lower cost.

A synthesis gas may comprise, in addition to hydrogen and carbon monoxide, carbon dioxide, water, methane, ethane, ethylene and so on. In this invention, no problem may arise when these components are present in a reaction atmosphere. A higher yield of propane and/or butane may be achieved when methane, ethane or ethylene is present in a reaction atmosphere.

### BRIEF DESCRIPTION OF THE DRAWINGS

FIG. 1 is a process flow diagram showing a main configuration in an example of an LPG producing apparatus suitable for conducting the process for LPG production according to this invention.

### DESCRIPTION OF THE MAIN SYMBOLS

11: a reformer
11a: a reforming catalyst (a catalyst for producing a synthesis gas)
12: a reactor for producing methanol
12a: a methanol synthesis catalyst
13: a reactor for producing a liquefied petroleum gas
13a: a catalyst for producing a liquefied petroleum gas
21, 22, 23, 24, 25, 26: lines.

### BEST MODE FOR CARRYING OUT THE INVENTION

1. Catalyst for Producing a Liquefied Petroleum Gas

Examples of a catalyst for producing a liquefied petroleum gas used in this invention include a catalyst comprising an olefin-hydrogenation catalyst component and a zeolite; for example, a catalyst in which an olefin-hydrogenation catalyst component is supported on a zeolite; and a mixed catalyst comprising a zeolite and a catalyst component in which an olefin-hydrogenation catalyst component is supported on a support such as silica. Other examples of a catalyst for producing a liquefied petroleum gas include a catalyst comprising a methanol synthesis catalyst and a zeolite; specifically, a catalyst comprising a Cu—Zn-based methanol synthesis catalyst and a USY-type zeolite in a ratio of the Cu—Zn-based methanol synthesis catalyst: the USY-type zeolite=1:5 to 2:1 (by weight); a catalyst comprising a Cu—Zn-based methanol synthesis catalyst and β-zeolite in a ratio of the Cu—Zn-based methanol synthesis catalyst: the β-zeolite=1:5 to 2:1 (by weight); and a catalyst comprising a Pd-based methanol synthesis catalyst and a β-zeolite in a ratio of the Pd-based methanol synthesis catalyst: the β-zeolite=1:5 to 2.5:1 (by weight).

Herein, an “olefin-hydrogenation catalyst component” means a compound which can act as a catalyst in a hydrogenation reaction of an olefin into a paraffin. Specific examples of an olefin-hydrogenation catalyst component include Fe, Co, Ni, Cu, Zn, Ru, Rh, Pd, Ir, Pt and so on. And a “methanol synthesis catalyst” means a compound which can act as a catalyst in the reaction of CO+2H₂→CH₃OH. In the above catalyst comprising a methanol synthesis catalyst and a zeolite, the methanol synthesis catalyst acts as an olefin-hydrogenation catalyst component. And a zeolite is those which can act as a catalyst in a condensation reaction of methanol into a hydrocarbon and/or a condensation reaction of dimethyl ether into a hydrocarbon.

In this invention, paraffin containing propane or butane as a main component (LPG) may be produced from at least one selected from the group consisting of methanol and dimethyl ether, and hydrogen, following the formula (I) shown below.

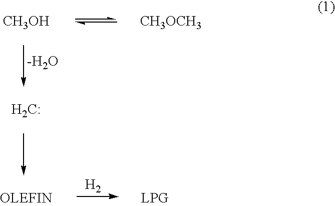

In this invention, methanol is dehydrated to generate a carbene (H₂C:) by a concerted catalysis of an acidic site and a basic site, which are at a spatial field inside a pore in a zeolite. And then, the carbene is polymerized to form an olefin containing propylene or butene as a main component. More specifically, it may be thought that ethylene is formed as a dimer; propylene is formed as a trimer or a reaction product with ethylene; and butylene is formed as a tetramer, a reaction product with propylene or a product of dimerization of ethylene.

In the olefin formation process, there would occur other reactions such as formation of dimethyl ether by dehydration-dimerization of methanol and formation of methanol by hydration of dimethyl ether.

And then, the formed olefin is hydrogenated by the catalysis of an olefin-hydrogenation catalyst component, to form a paraffin containing propane or butane as a main component, i.e., LPG.

For the above catalyst, any of Cu—Zn-based methanol synthesis catalysts known in the art can be used. Meanwhile, examples of a Pd-based methanol synthesis catalyst include a catalyst in which 0.1 to 10 wt % Pd is supported on a support such as silica; and a catalyst in which 0.1 to 10 wt % Pd and 5 wt % or less (excluding 0 wt %) at least one selected from the group consisting of alkali metals, alkaline earth metals and lanthanoid metals such as Ca are supported on a support such as silica.

Among them, a particularly preferable catalyst for producing a liquefied petroleum gas is a catalyst in which an olefin-hydrogenation catalyst component is supported on a zeolite; and a mixed catalyst comprising a zeolite and a catalyst component in which an olefin-hydrogenation catalyst component is supported on a support such as silica.

Specific examples of an olefin-hydrogenation catalyst component include Fe, Co, Ni, Cu, Zn, Ru, Rh, Pd, Ir, Pt and so on. The olefin-hydrogenation catalyst components may be used alone or in combination of two or more.

Among them, a preferable olefin-hydrogenation catalyst component is Pd or Pt, more preferably Pd. By using Pd and/or Pt as an olefin-hydrogenation catalyst component, the production amount of carbon monoxide and carbon dioxide as by-products can be more sufficiently reduced, while maintaining a high yield of propane and butane.

Pd and Pt may not be necessarily contained as a metal, but can be contained in the form of an oxide, a nitrate, a chloride or the like. In such a case, it is preferred that the catalyst may be subjected to, for example, reduction by hydrogen before the reaction, to convert Pd and/or Pt into metallic palladium and/or metallic platinum, for achieving higher catalytic activity.

The reduction treatment condition for activating Pd and/or Pt can be determined, depending on some factors such as the types of a supported palladium compound and/or a supported platinum compound, as appropriate.

A preferable catalyst in which an olefin-hydrogenation catalyst component is supported on a zeolite is Pd and/or Pt supported ZSM-5 or USY-type zeolite, more preferably Pd supported ZSM-5 or USY-type zeolite. By using ZSM-5 or USY-type zeolite as a zeolite for a support of Pd and/or Pt, a higher catalytic activity and a higher yield of propane and butane can be achieved, and furthermore the production amount of carbon monoxide and carbon dioxide as by-products can be more sufficiently reduced.

In the light of catalytic activity, Pd and/or Pt is preferably supported on a ZSM-5 or USY-type zeolite in a highly dispersed manner.

In this catalyst for producing a liquefied petroleum gas, the total amount of supported Pd and/or Pt is preferably 0.005 wt % or more, more preferably 0.01 wt % or more, particularly preferably 0.05 wt % or more, in the light of achieving a higher selectivity. And, the total amount of supported Pd and/or Pt is preferably 5 wt % or less, more preferably 1 wt % or less, particularly preferably 0.7 wt % or less, in the light of catalytic activity, dispersity and economical efficiency. By adjusting the total amount of supported Pd and/or Pt in a catalyst for producing a liquefied petroleum gas within the above range, propane and/or butane can be produced with a higher conversion, a higher selectivity and a higher yield.

A preferable ZSM-5, on which an olefin-hydrogenation catalyst component is supported, is high-silica ZSM-5, more specifically ZSM-5 with a Si/Al ratio (atomic ratio) of 20 to 100. By using ZSM-5 with a Si/Al ratio (atomic ratio) of 20 to 100, a higher catalytic activity and a higher yield of propane and butane can be achieved, and furthermore the production amount of carbon monoxide and carbon dioxide as by-products can be more sufficiently reduced. A Si/Al ratio (atomic ratio) of ZSM-5 is more preferably 70 or less, particularly preferably 60 or less.

A SiO₂/Al₂O₃ ratio of a USY-type zeolite is more preferably 5 or more, particularly preferably 15 or more. And, a SiO₂/Al₂O₃ ratio of a USY-type zeolite is preferably 70 or less, more preferably 50 or less, particularly preferably 40 or less, further preferably 25 or less.

The above catalyst for producing a liquefied petroleum gas may be a catalyst in which other components, in addition to Pd and/or Pt, are supported on ZSM-5 or USY-type zeolite, as long as the desired effects of the catalyst would not be impaired.

A catalyst for producing a liquefied petroleum gas in which an olefin-hydrogenation catalyst component is supported on a zeolite can be prepared by a known method such as an ion exchange method and an impregnation method. Sometimes, in comparison with a catalyst for producing a liquefied petroleum gas prepared by an impregnation method, a catalyst for producing a liquefied petroleum gas prepared by an ion exchange method may exhibit a higher catalytic activity, and thus may allow an LPG production reaction to proceed at a lower reaction temperature, and a higher selectivity for a hydrocarbon and a higher selectivity for propane and butane may be achieved.

A catalyst in which an olefin-hydrogenation catalyst component is supported on zeolite may be used, if necessary, after pulverization or molding. A molding method of a catalyst is not particularly limited, but is preferably a dry method including an extrusion and a tablet-compression.

A preferable mixed catalyst comprising a zeolite and a catalyst component in which an olefin-hydrogenation catalyst component is supported on a support is a mixed catalyst comprising a Pd-based catalyst component in which Pd is supported on a support and a USY-type zeolite. By using a USY-type zeolite, a higher catalytic activity and a higher yield of propane and butane can be achieved, and furthermore the production amount of carbon monoxide and carbon dioxide as by-products can be more sufficiently reduced.

A ratio of the Pd-based catalyst component to the USY-type zeolite (Pd-based catalyst component/USY-type zeolite; by weight) is preferably 0.1 or more, more preferably 0.3 or more. By adjusting a ratio of the Pd-based catalyst component to the USY-type zeolite (Pd-based catalyst component/USY-type zeolite; by weight) to 0.1 or more, a higher yield of LPG can be achieved.

A ratio of the Pd-based catalyst component to the USY-type zeolite (Pd-based catalyst component/USY-type zeolite; by weight) is preferably 1.5 or less, more preferably 1.2 or less, particularly preferably 0.8 or less. By adjusting a ratio of the Pd-based catalyst component to the USY-type zeolite (Pd-based catalyst component/USY-type zeolite; by weight) to 1.5 or less, a higher yield of LPG can be achieved, and furthermore the production amount of carbon monoxide, carbon dioxide and methane as by-products can be more sufficiently reduced. Moreover, by adjusting a ratio of the Pd-based catalyst component to the USY-type zeolite (Pd-based catalyst component/USY-type zeolite; by weight) to 0.8 or less, a further higher yield of LPG can be achieved, and the production amount of heavy hydrocarbons (C5 or more) as by-products can be more sufficiently reduced.

A ratio of the Pd-based catalyst component to the USY-type zeolite is not limited to the above range, and can be appropriately determined, depending on the amount of Pd in the Pd-based catalyst component, and the like.

A Pd-based catalyst component is Pd supported on a support. In the light of catalytic activity, Pd is preferably supported on a support in a highly dispersed manner.

The amount of supported Pd in a Pd-based catalyst component is preferably 0.1 wt % or more, more preferably 0.3 wt % or more. In the light of dispersibility and economical efficiency, the amount of supported Pd in a Pd-based catalyst component is preferably 5 wt % or less, more preferably 3 wt % or less. By adjusting the amount of supported Pd in a Pd-based catalyst component within the above range, propane and/or butane can be produced with a higher conversion, a higher selectivity and a higher yield.

A support for Pd-based catalyst component may be selected from known supports without limitation. Examples of a support include silica (silicon dioxide), alumina, silica-alumina, carbon (activated charcoal); and oxides of zirconium, titanium, cerium, lanthanum, iron or the like, and composite oxides containing two or more types of these metals, and composite oxides containing one or more types of these metals and one or more types of other metals.

Among them, a preferable support for Pd-based catalyst component is silica. By using silica as a support, propane and/or butane can be produced with a higher selectivity and a higher yield without producing carbon dioxide as a by-product.

A silica support preferably has a specific surface area of 450 m²/g or more, more preferably 500 m²/g or more. By using a silica having a specific surface area within the above range, higher catalytic activity can be achieved and propane and/or butane can be produced with a higher conversion and a higher yield.

The upper limit of a specific surface area of a silica support is not particularly restricted, but is generally about 1000 m²/g.

A specific surface area of silica can be determined, for example, by a BET method using N₂ as an adsorption gas and a fully automatic measuring apparatus for specific surface area and pore distribution (e.g., ASAP2010, Shimadzu Corporation).

A Pd-based catalyst component may be a catalyst component in which other components, in addition to Pd, are supported on a support, as long as the desired effects of the catalyst would not be impaired.

A Pd-based catalyst component, in which Pd is supported on a support (e.g. silica), can be prepared by a known method such as an impregnation method and a precipitation method.

Some of Pd-based catalyst components must be activated by reduction treatment before use, including those containing Pd as an oxide, nitrate or chloride. In this invention, it is not necessarily required to activate a Pd-based catalyst component by reduction treatment in advance. The Pd-based catalyst component can be activated by reduction treatment of the catalyst for producing a liquefied petroleum gas of this invention, before the beginning of the reaction, after producing the catalyst by mixing a Pd-based catalyst component and a USY-type zeolite, and then molding the mixture. The conditions of the reduction treatment can be determined, depending on some factors such as the type of the Pd-based catalyst component, as appropriate.

A USY-type zeolite to be used may be selected from USY-type zeolites containing a metal such as alkali metals, alkaline earth metals and transition metals; USY-type zeolites ion-exchanged with these metals or the like; and USY-type zeolites on which these metals or the like are supported. But a preferable USY-type zeolite is a proton-type zeolite. By using a proton-type USY-type zeolite having a suitable acid strength and a suitable acidity (acid concentration), higher catalytic activity can be achieved, and propane and/or butane can be produced with a higher conversion and a higher selectivity. Alternatively, Pd and/or Pt supported USY-type zeolite can be preferably used.

A SiO₂/Al₂O₃ ratio of a USY-type zeolite is more preferably 5 or more, particularly preferably 15 or more. By using a USY-type zeolite with a SiO₂/Al₂O₃ ratio of 5 or more, particularly preferably 15 or more, the production amount of carbon monoxide and carbon dioxide as by-products can be more sufficiently reduced, and a higher selectivity of propane and butane can be achieved.

And, a SiO₂/Al₂O₃ ratio of a USY-type zeolite is preferably 70 or less, more preferably 50 or less, particularly preferably 40 or less, further preferably 25 or less. By using a USY-type zeolite with a SiO₂/Al₂O₃ ratio of 70 or less, further preferably 25 or less, a higher conversion of methanol and/or dimethyl ether can be achieved, and the production amount of methane as by-products can be more sufficiently reduced, and a higher selectivity of propane and butane can be achieved.

A mixed catalyst comprising a Pd-based catalyst component and a USY-type zeolite, that is a catalyst for producing a liquefied petroleum gas, is prepared by separately preparing a Pd-based catalyst component and a USY-type zeolite, and homogeneously mixing them, and then, if necessary, molding the mixture. A procedure of mixing and molding these catalyst components is not particularly limited, but is preferably a dry method. When mixing and molding these catalyst components by a wet method, there may occur a compound transfer between these catalyst components, for example, neutralization due to transfer of a basic component in a Pd-based catalyst component to an acidic site in a USY-type zeolite, leading to the change of a property optimized for each function of these catalyst components, and the like. Examples of a molding method of a catalyst include an extrusion and a tablet-compression.

A catalyst for producing a liquefied petroleum gas may comprise other additive components as long as its intended effect would not be impaired. For example, any of the above catalysts may be diluted with quartz sand and then used.

When the reaction is conducted with a fixed bed, in the catalyst layer comprising a catalyst for producing a liquefied petroleum gas, the composition may change in regard to the direction of flowing of the starting gas. The catalyst layer may consist of, for example, a former catalyst layer comprising a zeolite largely, and a latter catalyst layer comprising a catalyst component in which an olefin-hydrogenation catalyst component is supported on a support such as silica, or a methanol synthesis catalyst component largely, in the direction of flowing of the starting gas.

2. Process for Producing a Liquefied Petroleum Gas

In this invention, a paraffin comprising propane or butane, preferably propane, as a main component is produced by reacting at least one selected from the group consisting of methanol and dimethyl ether with hydrogen using at least one of the catalysts for producing a liquefied petroleum gas as described above. In this process, a gas fed into a reactor (a raw material gas or a starting gas) comprises at least one selected from the group consisting of methanol and dimethyl ether, hydrogen, and carbon monoxide, preferably at least one selected from the group consisting of methanol and dimethyl ether, and a synthesis gas. Subsequently, water, a low-boiling component such as hydrogen, and a high-boiling component is separated from the lower-paraffin-containing gas produced, as necessary, to obtain a liquefied petroleum gas (LPG). If necessary, the gas may be pressurized and/or cooled so as to obtain a liquefied petroleum gas.

Hydrogen and carbon monoxide, or a synthesis gas to be used may be the unreacted raw material of the methanol synthesis reaction (CO+2H₂→CH₃OH) or the dimethyl ether synthesis reaction (3CO+3H₂→CH₃OCH₃+CO₂). A reaction product gas of the methanol synthesis reaction or the dimethyl ether synthesis reaction may be fed into a reactor as a raw material gas, without separating hydrogen and carbon monoxide, which are unreacted raw materials. Alternatively, a gas fed into a reactor may be a gas obtained by separating an excessive amount of hydrogen and/or carbon monoxide from the reaction product gas of the methanol synthesis reaction or the dimethyl ether synthesis reaction. A gas fed into a reactor may be a gas obtained by separating hydrogen and carbon monoxide from the reaction product gas of the methanol synthesis reaction or the dimethyl ether synthesis reaction, and then adding a suitable amount of hydrogen and carbon monoxide to the resulting gas.

In the process for producing LPG according to this invention, a reaction raw material may be methanol or dimethyl ether alone, or may be a mixture of methanol and dimethyl ether. When using a mixture of methanol and dimethyl ether as a reaction raw material, a ratio of methanol to dimethyl ether is not particularly limited, and can be appropriately determined.

The reaction can be conducted in a fixed bed, a fluidized bed or a moving bed. The reaction conditions such as a composition of a starting gas, a reaction temperature, a reaction pressure and a contact time with a catalyst can be appropriately determined, depending on a kind of a catalyst to be used, and the like. For example, the LPG production reaction may be carried out under the following conditions.

A gas fed into a reactor comprises at least one selected from the group consisting of methanol and dimethyl ether, hydrogen, and carbon monoxide. A gas fed into a reactor may contain other components which a synthesis gas may generally contain; specifically, carbon dioxide, water vapor, methane, ethane, ethylene and so on.

When a reaction raw material is methanol, a content of hydrogen in a gas fed into a reactor is preferably 0.5 mole or more, more preferably 0.8 moles or more per 1 mole of methanol, in the light of improving a hydrogenation rate and reducing deterioration of a catalyst. In the light of productivity and economical efficiency, a content of hydrogen in a gas fed into a reactor is preferably 2 moles or less, more preferably 1.2 moles or less per 1 mole of methanol.

When a reaction raw material is dimethyl ether, a content of hydrogen in a gas fed into a reactor is preferably 1 mole or more, more preferably 1.5 moles or more per 1 mole of dimethyl ether, in the light of improving a hydrogenation rate and reducing deterioration of a catalyst. In the light of productivity and economical efficiency, a content of hydrogen in a gas fed into a reactor is preferably 3 moles or less, more preferably 2.4 moles or less per 1 mole of dimethyl ether.

When a reaction raw material is a mixture of methanol and dimethyl ether, a content of hydrogen in a gas fed into a reactor is preferably within the same range as the above preferable range when a reaction raw material is methanol and the above preferable range when a reaction raw material is dimethyl ether. And, this preferable range can be calculated based on a ratio of methanol to dimethyl ether.

A content of carbon monoxide in a gas fed into a reactor can be appropriately determined, and is preferably 18 to 30 mol %. A content of carbon monoxide in a gas fed into a reactor is more preferably 20 mol % or more. In the light of productivity, a content of carbon monoxide in a gas fed into a reactor is more preferably 25 mol % or less.

When using a synthesis gas as the source of hydrogen, the feed ratio of the synthesis gas to at least one selected from the group consisting of methanol and dimethyl ether may be appropriately determined so that a composition of a starting gas may be within the above range. If necessary, hydrogen may be added to the gas consisting of methanol and/or dimethyl ether, and a synthesis gas.

In the light of achieving a higher catalytic activity, a reaction temperature is preferably 300° C. or higher, more preferably 320° C. or higher. In the light of achieving a higher selectivity for a hydrocarbon and a higher selectivity for propane and butane, as well as a long catalyst life, a reaction temperature is preferably 470° C. or lower, more preferably 450° C. or lower, particularly preferably 400° C. or lower.

In the light of achieving a higher activity and good operability of an apparatus, a reaction pressure is preferably 0.1 MPa or higher, more preferably 0.15 MPa or higher. In the light of economical efficiency and safety, a reaction pressure is preferably 3 MPa or lower, more preferably 2.5 MPa or lower.

Furthermore, according to this invention, LPG can be produced under a further lower pressure. Specifically, LPG can be produced from at least one selected from the group consisting of methanol and dimethyl ether, and hydrogen under a pressure of lower than 1 MPa, particularly 0.6 MPa or lower.

A gas space velocity of methanol and/or dimethyl ether is preferably 1500 hr⁻¹ or more, more preferably 1800 hr⁻¹ or more, in the light of economical efficiency. In addition, a gas space velocity of methanol and/or dimethyl ether is preferably 60000 hr⁻¹ or less, more preferably 30000 hr⁻¹ or less, in the light of achieving a higher activity and a higher selectivity for propane and butane.

At least one selected from the group consisting of methanol and dimethyl ether, hydrogen, and carbon monoxide may be mixed, and then fed to a reactor or, alternatively, these may be fed to a reactor separately.

A gas fed into a reactor can be dividedly fed to the reactor so as to control a reaction temperature.

The reaction can be conducted in a fixed bed, a fluidized bed, a moving bed or the like, and can be preferably selected, taking both of control of a reaction temperature and a regeneration method of the catalyst into account. For example, a fixed bed may include a quench type reactor such as an internal multistage quench type, a multitubular type reactor, a multistage type reactor having a plurality of internal heat exchangers or the like, a multistage cooling radial flow type, a double pipe heat exchange type, an internal cooling coil type, a mixed flow type, and other types of reactors.

When used, a catalyst for producing a liquefied petroleum gas can be diluted with silica, alumina or an inert and stable heat conductor for controlling a temperature. In addition, when used, a catalyst for producing a liquefied petroleum gas can be applied to the surface of a heat exchanger for controlling a temperature.

A reaction product gas thus produced (a lower-paraffin-containing gas) comprises a hydrocarbon containing propane or butane as a main component. In the light of liquefaction properties, it is preferable that the total content of propane and butane is higher in a lower-paraffin-containing gas. Furthermore, a lower-paraffin-containing gas produced preferably contains more propane in comparison with butane, in the light of inflammability and vapor pressure properties.

A lower-paraffin-containing gas produced generally comprises water; a low-boiling component having a lower boiling point or a lower sublimation point than the boiling point of propane; and a high-boiling component having a higher boiling point than the boiling point of butane. Examples of a low-boiling component include carbon monoxide and carbon dioxide; hydrogen, which is an unreacted starting material; and ethane and methane, which are by-products. Examples of a high-boiling component include high-boiling paraffins (e.g., pentane, hexane and so on), which are by-products.

Thus, water, a low-boiling component and a high-boiling component are, as necessary, separated from a lower-paraffin-containing gas produced, so as to obtain a liquefied petroleum gas (LPG) comprising propane or butane as a main component. If necessary, methanol and/or dimethyl ether, which are unreacted starting materials, are also separated from a lower-paraffin-containing gas by a known method.

Separation of water, a low-boiling component or a high-boiling component can be conducted in accordance with a known method.

Water can be separated by, for example, liquid-liquid separation.

A low-boiling component can be separated by, for example, gas-liquid separation, absorption separation or distillation; more specifically, gas-liquid separation at an ambient temperature under increased pressure, absorption separation at an ambient temperature under increased pressure, gas-liquid separation with cooling, absorption separation with cooling, or combination thereof. Alternatively, for this purpose, membrane separation or adsorption separation can be conducted, or these in combination with gas-liquid separation, absorption separation or distillation can be conducted. A gas recovery process commonly employed in an oil factory (described in “Oil Refining Processes”, ed. The Japan Petroleum Institute, Kodansha Scientific, 1998, pp. 28-32) can be applied to separation of a low-boiling component.

A preferable method of separation of a low-boiling component is an absorption process where a liquefied petroleum gas comprising propane or butane as a main component is absorbed into an absorbent liquid such as a high-boiling paraffin gas having a higher boiling point than butane, and a gasoline.

A high-boiling component can be separated by, for example, gas-liquid separation, absorption separation or distillation.

The separation conditions may be determined as appropriate in accordance with a known method.

For consumer use, it is preferable that a content of a low-boiling component in the LPG is reduced to 5 mol % or less (including 0 mol %) by separation, for example, in the light of safety in use.

If necessary, the gas may be pressurized and/or cooled so as to obtain a liquefied petroleum gas.

The total content of propane and butane in the LPG thus produced may be 90% or more, more preferably 95% or more (including 100%) on the basis of carbon. And a content of propane in the LPG produced may be 50% or more, more preferably 60% or more, particularly preferably 65% or more (including 100%) on the basis of carbon. Thus, according to this invention, LPG having a composition suitable for a propane gas, which is widely used as a fuel for household and business use, can be produced.

3. Process for Producing a Liquefied Petroleum Gas from a Carbon-Containing Starting Material

At present, methanol and dimethyl ether, which are used as a starting material in this invention, are produced in an industrial scale.

Methanol is produced, for example, as follows.

First, a synthesis gas is produced by reacting a natural gas (methane) with at least one selected from the group consisting of H₂O, O₂ and CO₂ in the presence of a reforming catalyst such as an Ni-based catalyst, if necessary, after removing a catalyst poisoning component such as sulfur and a sulfur compound from a natural gas (methane) (devulcanization and the like). A water-vapor reforming method, a complex reforming method and an autothermal reforming method of a natural gas (methane) are well known as a process for producing a synthesis gas.

And, a synthesis gas may be also produced by reacting a carbon-containing starting material other than a natural gas with at least one selected from the group consisting of H₂O, O₂ and CO₂ in accordance with a known method. A carbon-containing substance which can react with at least one selected from the group consisting of H₂O, O₂ and CO₂ to form H₂ and CO, can be used as a carbon-containing starting material. For example, a lower hydrocarbon such as ethane, a naphtha, a coal, and the like can be used.

Next, methanol is produced from the synthesis gas by reacting carbon monoxide with hydrogen in the presence of a methanol synthesis catalyst. When using a Cu—Zn-based catalyst (a composite oxide containing Cu and Zn) such as a Cu—Zn—Al composite oxide and a Cu—Zn—Cr composite oxide as a methanol synthesis catalyst, the reaction is generally carried out at a reaction temperature of about 230 to 300° C. and under a reaction pressure of about 2 to 10 MPa. When using a Zn—Cr-based catalyst (a composite oxide containing Zn and Cr) as a methanol synthesis catalyst, the reaction is generally carried out at a reaction temperature of about 250 to 400° C. and under a reaction pressure of about 10 to 60 MPa. Thus, methanol can be synthesized.

Dimethyl ether is produced by, for example, dehydration reaction of methanol using a solid acid catalyst such as aluminum phosphate.

A process for producing dimethyl ether from a synthesis gas directly, not via methanol, is being put to practical use. In the process, dimethyl ether can be produced by reacting carbon monoxide with hydrogen at a reaction temperature of about 230 to 280° C. and under a reaction pressure of about 3 to 7 MPa in the presence of a mixed catalyst of a methanol synthesis catalyst and a methanol dehydration catalyst, for example, a mixed catalyst comprising a methanol synthesis catalyst and a methanol dehydration catalyst in a ratio of the methanol synthesis catalyst: the methanol dehydration catalyst=1:2 to 2:1 (by weight), using a slurry phase reactor.

Thus, methanol and dimethyl ether are produced from a synthesis gas. In this invention, a synthesis gas can be also used as the source of hydrogen for LPG production.

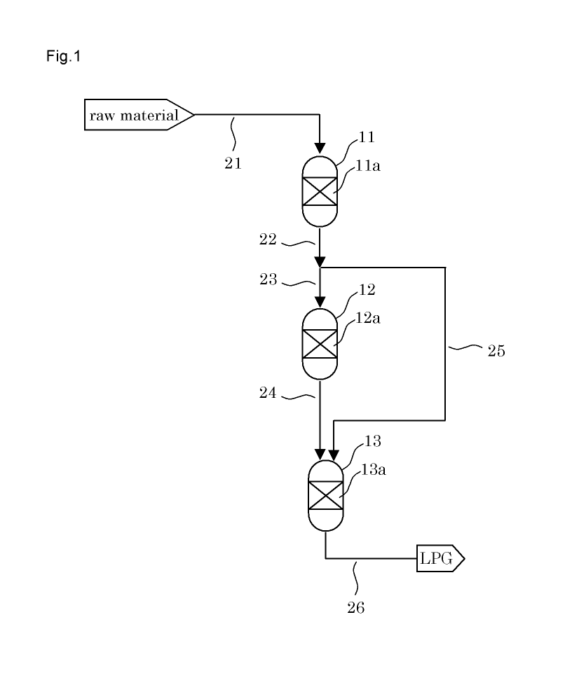

FIG. 1 shows an embodiment of an LPG production apparatus suitable for carrying out a process for producing a liquefied petroleum gas (LPG) according to this invention wherein LPG is produced from a carbon-containing starting material such as a natural gas, via a synthesis gas, and methanol and/or dimethyl ether.

First, a natural gas (methane) as a carbon-containing starting material is fed into a reformer 11 via a line 21. And, as necessary, oxygen, steam or carbon dioxide is also fed into the line 21 (not shown). In the reformer 11, there is a reforming catalyst layer comprising a reforming catalyst (a catalyst for producing a synthesis gas) 11a. The reformer 11 also has a heating means for supplying heat required for reforming (not shown). In the reformer 11, methane is reformed in the presence of the reforming catalyst to produce a synthesis gas containing hydrogen and carbon monoxide. A synthesis gas can be produced in accordance with a known method including the method described above.

Part of the synthesis gas thus produced is fed into a reactor for producing a liquefied petroleum gas 13 via lines 22 and 25 as the source of hydrogen for LPG production; while the rest of the synthesis gas is fed into a reactor for producing methanol 12 via lines 22 and 23. In the reactor 12, there is a catalyst layer comprising a methanol synthesis catalyst 12a. In the reactor 12, methanol is synthesized from the synthesis gas in the presence of the methanol synthesis catalyst. Methanol can be synthesized in accordance with a known method including the method described above.

After purification, as necessary, or without purification, the methanol thus produced is fed into a reactor for producing a liquefied petroleum gas 13 via a line 24. And, as the source of hydrogen, the synthesis gas is fed into a reactor for producing a liquefied petroleum gas 13 from the reformer 11 via lines 22 and 25. In the reactor 13, there is a catalyst layer comprising a catalyst for producing a liquefied petroleum gas 13a. In the reactor 13, a hydrocarbon gas containing propane or butane as a main component (a lower-paraffin-containing gas) is produced from methanol and hydrogen in the presence of the catalyst for producing a liquefied petroleum gas. LPG can be produced in accordance with the above process.

The hydrocarbon gas thus produced is pressurized and cooled, after optional removal of water, a low-boiling component including hydrogen, and a high-boiling component by, for example, gas-liquid separation, and LPG, which is a product, is obtained from a line 26.

The component(s) separated from the lower-paraffin-containing gas can be removed outside the system, and can be recycled to any process as described above. For example, carbon monoxide and hydrogen separated in this step can be recycled to the lower-paraffin production process or the methanol production process. Carbon monoxide and carbon dioxide separated in this step can be recycled to the synthesis gas production process.

For the purpose of recycling a separated component, a known technique, e.g., appropriately providing a recycle line with a pressurization means can be employed.

The LPG production apparatus may be, as necessary, provided with a booster, a heat exchanger, a valve, an instrumentation controller and so on, which are not shown.

A dimethyl ether synthesis catalyst can be substituted for a methanol synthesis catalyst 12a. In this case, dimethyl ether is synthesized from the synthesis gas in the reactor 12, and then a hydrocarbon gas containing propane or butane as a main component (a lower-paraffin-containing gas) is produced from the dimethyl ether in the reactor 13. Dimethyl ether can be synthesized in accordance with a known method including the method described above.

In the above process for producing LPG, a shift reactor may be placed downstream of a reformer, which is a reactor for producing a synthesis gas, so that a synthesis gas composition can be adjusted by a shift reaction (CO+H₂O→CO₂+H₂).

Thus, according to this invention, LPG containing propane or butane as a main component may be produced from a carbon-containing starting material such as a natural gas, via a synthesis gas, and methanol and/or dimethyl ether.

### EXAMPLES

The following will describe the present invention in more detail with reference to Examples. However, the present invention is not limited to these Examples.

### Example 1

### Preparation of a Catalyst

A mechanically pulverized proton-type ZSM-5 with a Si/Al ratio (atomic ratio) of 20, produced by Tosoh Corporation, was used as a zeolite for a support of an olefin-hydrogenation catalyst component, Pd. And, 0.5 wt % of Pd was supported on the ZSM-5 by an ion exchange method as follows.

First, 0.0825 g of palladium chloride (purity: >99 wt %) was dissolved in 10 mL of a 12.5 wt % aqueous ammonia solution at 40 to 50° C. And then, 150 mL of ion-exchanged water was added to the resulting solution to obtain a Pd-containing solution. 10 g of ZSM-5 zeolite was added to the obtained Pd-containing solution, and the mixture was heated and stirred at 60 to 70° C. for 6 hours. After the ion-exchange process, the resulting material was repeatedly filtrated and washed with ion-exchanged water until no chloride ions were observed in a filtrate.

Then, the Pd ion-exchanged ZSM-5 was dried at 120° C. for 12 hours, and calcined at 500° C. in an air for 2 hours. Subsequently, it was mechanically pulverized, and then molded by a tablet-compression and sized to give a granular catalyst for producing a liquefied petroleum gas (Pd-ZSM-5) having an average particle size of 1 mm.

(Production of Lpg)

In a tubular reactor with an inner diameter of 6 mm was placed 1 g of the catalyst prepared as above, and the catalyst was reduced under a hydrogen stream at 400° C. for 3 hours before the beginning of the reaction.

After reduction treatment of the catalyst, a starting gas with a composition of dimethyl ether: a synthesis gas (CO:H₂=1:2)=1:3 was passed through the catalyst layer at a reaction temperature of 350° C., a reaction pressure of 2.1 MPa and a gas space velocity of dimethyl ether of 2000 hr⁻¹ (W/F=9.0 g h/mol) to carry out the LPG production reaction. The content of carbon monoxide in the starting gas is 25 mol %, and the ratio of hydrogen to dimethyl ether is H₂/DME=2 (molar ratio).

Gas chromatographic analysis of the product indicated that, after three hours from the beginning of the reaction, a conversion of dimethyl ether was 100%, and a conversion of dimethyl ether to a hydrocarbon was 104.1% on the basis of carbon. This is because carbon monoxide in the starting gas was also converted into hydrocarbons. The produced hydrocarbon gas contained propane and butane in 47.1% on the basis of carbon, and a conversion of dimethyl ether to propane and butane was 49.0% on the basis of carbon.

The results are shown in Table 1.

### Comparative Example 1

The LPG production reaction was carried out in the same way as Example 1, except that a starting gas consisting of 25 mol % of dimethyl ether and 75 mol % of hydrogen (H₂/DME=3 (molar ratio)) was used, and the gas space velocity of dimethyl ether was equal to that in Example 1.

Gas chromatographic analysis of the product indicated that, after three hours from the beginning of the reaction, a conversion of dimethyl ether was 100%, and a conversion of dimethyl ether to a hydrocarbon was 98.2% on the basis of carbon. The produced hydrocarbon gas contained propane and butane in 50.6% on the basis of carbon, and a conversion of dimethyl ether to propane and butane was 49.7% on the basis of carbon.

The results are shown in Table 1.

TABLE 1

| | | Comparative |
|---|---|---|
| | Example 1 | Example 1 |
| Reaction temperature (° C.) | 350 | 350 |
| DME conversion (%) | 100.0 | 100.0 |
| Hydrocarbon yield (%) | 104.1 | 98.2 |
| Product composition (%) | | |
| C1 (methane) | 2.0 | 1.8 |
| C2 (ethane) | 26.3 | 29.2 |
| C3 (propane) | 27.1 | 33.4 |
| C4 (butane) | 20.0 | 17.3 |
| C5 (pentane) | 12.5 | 11.3 |
| C6 (hexane) | 9.5 | 7.2 |
| C7 (heptane) | 2.7 | — |
| C3 + C4 yield (%) | 49.0 | 49.7 |

### Example 2

Using the catalyst prepared in the same way as Example 1 (Pd-ZSM-5), the LPG production reaction was carried out in the same way as Example 1, except that a reaction temperature was 375° C.

Gas chromatographic analysis of the product indicated that, after three hours from the beginning of the reaction, a conversion of dimethyl ether was 100%, and a conversion of dimethyl ether to a hydrocarbon was 106.0% on the basis of carbon. This is because carbon monoxide in the starting gas was also converted into hydrocarbons. The produced hydrocarbon gas contained propane and butane in 47.7% on the basis of carbon, and a conversion of dimethyl ether to propane and butane was 50.5% on the basis of carbon.

The results are shown in Table 2.

### Comparative Example 2

The LPG production reaction was carried out in the same way as Example 2, except that a starting gas consisting of 25 mol % of dimethyl ether and 75 mol % of hydrogen (H₂/DME=3 (molar ratio)) was used, and the gas space velocity of dimethyl ether was equal to that in Example 2.

Gas chromatographic analysis of the product indicated that, after three hours from the beginning of the reaction, a conversion of dimethyl ether was 100%, and a conversion of dimethyl ether to a hydrocarbon was 99.2% on the basis of carbon. The produced hydrocarbon gas contained propane and butane in 50.9% on the basis of carbon, and a conversion of dimethyl ether to propane and butane was 50.5% on the basis of carbon.

The results are shown in Table 2.

TABLE 2

| | | Comparative |
|---|---|---|
| | Example 2 | Example 2 |
| Reaction temperature (° C.) | 375 | 375 |
| DME conversion (%) | 100.0 | 100.0 |
| Hydrocarbon yield (%) | 106.0 | 99.2 |
| Product composition (%) | | |
| C1 (methane) | 1.6 | 2.1 |
| C2 (ethane) | 26.3 | 27.6 |
| C3 (propane) | 29.2 | 29.7 |
| C4 (butane) | 18.5 | 21.2 |
| C5 (pentane) | 10.9 | 12.6 |
| C6 (hexane) | 11.6 | 5.8 |
| C7 (heptane) | 2.0 | 1.0 |
| C3 + C4 yield (%) | 50.5 | 50.5 |

### INDUSTRIAL APPLICABILITY

As described above, according to this invention, a hydrocarbon containing propane or butane as a main component, i.e., a liquefied petroleum gas (LPG) can be economically produced from methanol and/or dimethyl ether.

1. A process for producing a liquefied petroleum gas containing propane or butane as a main component, comprising a step of:
producing a liquefied petroleum gas by passing a raw material gas comprising at least one selected from the group consisting of methanol and dimethyl ether, hydrogen, and carbon monoxide through a catalyst layer comprising a catalyst for producing a liquefied petroleum gas.

2. A process for producing a liquefied petroleum gas containing propane or butane as a main component, comprising a step of:
producing a liquefied petroleum gas by passing a raw material gas comprising at least one selected from the group consisting of methanol and dimethyl ether, and a synthesis gas through a catalyst layer comprising a catalyst for producing a liquefied petroleum gas.

3. A process for producing a liquefied petroleum gas according to claim 1, wherein
the content of carbon monoxide in the raw material gas is 18 to 30 mol %; and
the content of hydrogen in the raw material gas is 0.5 to 2 mole per 1 mole of methanol, or 1 to 3 mole per 1 mole of dimethyl ether.

4. A process for producing a liquefied petroleum gas according to claim 1, wherein the catalyst for producing a liquefied petroleum gas comprises an olefin-hydrogenation catalyst component and a zeolite.

5. A process for producing a liquefied petroleum gas according to claim 4, wherein the olefin-hydrogenation catalyst component is at least one selected from the group consisting of Fe, Co, Ni, Cu, Zn, Ru, Rh, Pd, Ir and Pt.

6. A process for producing a liquefied petroleum gas according to claim 5, wherein the catalyst for producing a liquefied petroleum gas is a catalyst in which Pd and/or Pt is supported on ZSM-5 or USY-type zeolite.

7. A process for producing a liquefied petroleum gas according to claim 6, wherein a Si/Al ratio (atomic ratio) of the ZSM-5 is 20 to 100, or a SiO₂/Al₂O₃ ratio of the USY-type zeolite is 5 to 70.

8. A process for producing a liquefied petroleum gas according to claim 6, wherein the total amount of supported Pd and/or Pt in the catalyst for producing a liquefied petroleum gas is 0.005 to 5% by weight.

9. A process for producing a liquefied petroleum gas according to claim 5, wherein the catalyst for producing a liquefied petroleum gas comprises a Pd-based catalyst component in which Pd is supported on a support; and a USY-type zeolite.

10. A process for producing a liquefied petroleum gas according to claim 9, wherein a ratio (by weight) of the Pd-based catalyst component to the USY-type zeolite is 0.1 to 1.5 (Pd-based catalyst component/USY-type zeolite).

11. A process for producing a liquefied petroleum gas according to claim 9, wherein the amount of supported Pd in the Pd-based catalyst component is 0.1 to 5% by weight.

12. A process for producing a liquefied petroleum gas according to claim 9, wherein the support for the Pd-based catalyst component is silica.

13. A process for producing a liquefied petroleum gas according to claim 9, wherein the USY-type zeolite has a SiO₂/Al₂O₃ ratio of 5 to 70.

14. A process for producing a liquefied petroleum gas, comprising:
(I) a step of producing a synthesis gas from a carbon-containing starting material and at least one selected from the group consisting of H₂O, O₂ and CO₂;
(II) a step of producing methanol wherein a methanol-containing gas is produced from the synthesis gas obtained in the step of producing a synthesis gas, using a methanol synthesis catalyst; and
(III) a step of producing a liquefied petroleum gas wherein a liquefied petroleum gas containing propane or butane as a main component is produced from the methanol-containing gas obtained in the step of producing methanol and the synthesis gas obtained in the step of producing a synthesis gas, using a catalyst for producing a liquefied petroleum gas.

15. A process for producing a liquefied petroleum gas, comprising:
(I) a step of producing a synthesis gas from a carbon-containing starting material and at least one selected from the group consisting of H₂O, O₂ and CO₂;
(II) a step of producing dimethyl ether wherein a dimethyl ether-containing gas is produced from the synthesis gas obtained in the step of producing a synthesis gas, using a dimethyl ether synthesis catalyst; and
(III) a step of producing a liquefied petroleum gas wherein a liquefied petroleum gas containing propane or butane as a main component is produced from the dimethyl ether-containing gas obtained in the step of producing dimethyl ether and the synthesis gas obtained in the step of producing a synthesis gas, using a catalyst for producing a liquefied petroleum gas.
